(19) United States
(12) Patent Application Publication
(10) Pub. No.: US 2012/0204276 A1
(43) Pub. Date: Aug. 9, 2012

(54) METHODS RELATED TO A MUTATION IN COMPLEMENT FACTOR H-RELATED PROTEIN 5 IN PATIENTS WITH GLOMERULONEPHRITIS

(75) Inventors: Daniel Gale, London (GB); Patrick Maxwell, London (GB); Matthew Pickering, London (GB); Elena Goicoechea De Jorge, London (GB)

(73) Assignee: UCL BUSINESS PLC, London (GB)

(21) Appl. No.: 13/382,831

(22) PCT Filed: Jul. 6, 2010
(86) PCT No.: PCT/GB2010/001295

§ 371 Date: Apr. 25, 2012

(30) Foreign Application Priority Data
Jul. 6, 2009 (GB) 0911717.7

(51) Int. Cl. A61K 38/02; A61K 31/7088; C07K 14/47; C12N 15/12; C12N 15/63; C12N 5/10; A01K 67/027; A61K 49/00; G01N 33/566; C12N 1/21; C12N 1/19; A61P 37/06; A61P 19/02; A61P 1/00; A61P 29/00; A61P 9/00; A61P 25/16; A61P 25/28; A61P 11/06; A61P 25/00; C12Q 1/68

(52) U.S. Cl. 800/3; 435/6.12; 514/44 R; 530/350; 536/23.5; 435/320.1; 435/348; 800/9; 436/501; 435/325; 435/252.3; 435/254.2; 435/365; 514/15.4; 514/16.6; 514/16.4; 514/1.1; 514/17.7; 514/17.8; 514/1.7; 514/17.9

(57) ABSTRACT

The invention relates to methods of regulating complement. In particular, the inventors have identified a relationship between a particular gene, CFHR5, and irregularities in complement regulation. The invention provides a method for diagnosing a complement related disease, comprising identifying a mutation in the CFHR5 gene in a sample obtained from a subject.

### FIELD OF THE INVENTION

The invention relates to methods of regulating complement. In particular, the inventors have identified a relationship between a particular gene, CFHR5, and irregularities in complement regulation.

### BACKGROUND TO THE INVENTION

Complement is a central element of the innate immune system. Its disordered regulation can contribute to a wide range of clinical conditions including age related macular degeneration, hemolytic uremic syndrome and renal diseases including glomerulonephritis, in which there is commonly deposition of complement components within the glomerulus. The inventors have identified multiple families in which an internal duplication in the Complement Factor H Related 5 (CFHR5) gene is associated with C3 glomerulonephritis and renal failure. The mutant protein is present in serum and has reduced affinity for tissue-bound complement. These findings implicate CFHR5 as an important regulator of complement in humans.

The complement alternate pathway (AP) is a phylogenetically ancient component of the innate immune system which is present in all vertebrates that have been studied and some protochordates.^{1,2} The meticulous study of humans with inherited or acquired complement abnormalities has significantly advanced the understanding of the immunobiology of this system³ and has demonstrated that its dysregulation underlies a range of disorders affecting diverse organ systems and presenting to physicians across a range of specialities.

For reasons that are not yet fully understood, the kidney appears particularly sensitive to the effects of complement activation. Glomerulonephritis is commonly associated with deposition of electron dense material in the glomerulus which contains complement components, including C3. This is almost always driven by immunoglobulins which are also present in the glomerular deposits. The reasons why some individuals develop severe glomerular injury in the setting of increased immunoglobulin production while the great majority do not are poorly understood, but insight into the role of complement in this process has been provided by recognition of rare cases in which complement is deposited in the absence of immunoglobulins. These have established that inherited or acquired dysregulation of the AP is sufficient to cause disease.⁴

AP dysregulation due to mutations or polymorphic variants in complement regulatory genes (including complement factor H (CFH), factor I (CFI) or membrane cofactor protein (CD46)), or the complement activating genes C3 and factor B (Bf)) has been implicated in cardiovascular disease^{5,7}, age-related macular degeneration (AMD),⁸ the HELLP syndrome of pregnancy,⁹ atypical hemolytic uremic syndrome (aHUS),^{8,10,11} and the renal diseases dense deposit disease (DDD)^{4,12 and} C3 glomerulonephritis (C3GN)^{8,13} which are characterised by glomerular C3 deposition in the absence of immunoglobulin. Importantly, AP dysregulation in these renal conditions may also be acquired: C3 nephritic factor (C3NeF), an antibody that potentiates AP activation, is associated with DDD and C3GN,¹² and anti-CFH antibodies have been associated with aHUS and DDD.^{14,15} C3GN is a recently recognized heterogeneous disorder which may present with isolated mesangial deposits of C3 with little glomerular inflammation or can be associated with sub-endothelial electron dense glomerular basement membrane (GBM) C3 deposits together with membranoproliferative glomerulonephritis¹³. Here we describe autosomal dominant inheritance of C3GN in families from Cyprus.

The disease segregates with a heterozygous internal duplication of complement factor H related 5 gene (CFHR5) and we show that this mutation reduces the ability of CFHR5 to interact with membrane-bound C3. Together, these findings demonstrate a novel facet of complement regulation in humans.

### SUMMARY OF THE INVENTION

According to the invention, there is provided a method for diagnosing a complement related disease, comprising identifying a mutation in the CFHR5 gene in a sample obtained from a subject.

The mutation may be any mutation within the gene, such as a point mutation such as the substitution, deletion or insertion of a single nucleotide. Alternatively, more than one nucleotide may be substituted, deleted or inserted. Much larger mutations are also envisaged, such as the deletion or repeat of entire exons, multiple exons; deletion of the entire gene or fusion of the gene with another gene (for instance due to non-homologous recombination). Mutations may include duplications of exon 2 or exon 3 or both. Mutations resulting in the incorrect translation of the gene, such as those introducing a shift in the reading frame or the insertion of a stop codon, may also be present. One example mutation is the duplication of the sequence, or a substantial part such as 80, 85, 90 or 95% of the sequence, shown in bold in FIG. 5.

The term complement related disease is used to mean any disease, disorder or syndrome in which complement is incorrectly regulated. Complement may be inappropriately activated or may not be activated when required. Alternatively, the magnitude of any complement response may be inappropriate. Complement related diseases include some renal diseases, and certain autoimmune disorders. In particular, complement related diseases include: Renal disorders known to be due to complement dysregulation:

a. C3 glomerulopathy
b. Haemolytic uraemic syndrome
c. Dense deposit disease (also known as MPGN Type II or C3Neph)

Renal diseases in which complement dysregulation plays an important role (as a consequence of antibody activation):

a. IgA nephropathy
b. Mesangiocapillary (membranoproliferative) glomerulonephritis (MPGN) including that seen in systemic disorders such as:
i. Hepatitis B
ii. Hepatitis C
iii. Chronic bacterial infections (eg tuberculosis, chronic infected ulcers and abscesses)
c. Autoimmune disorders (eg systemic lupus erythematosus, antiphospholipid syndrome)
d. Renal allograft nephropathy/rejection
e. ANCA associated vasculitis,

Non-renal disorders in which complement deposition or activation is a feature:

a. Age related macular degeneration
b. Thrombotic thrombocytopaenic purpura (TTP)
c. HELLP syndrome of pregnancy
d. Paroxysmal Nocturnal Haemoglobinuria

Non-renal disorders in which complement deposition is seen histologically, in experimental systems, or there is population genetic evidence for a role of complement regulators in modifying risk:

a. Autoimmune disorders
i. Rheumatoid arthritis
ii. Myositis/myocarditis
iii. Pemphigoid/pemphigus/epidermolysis bullosa
iv. Crohn's disease/ulcerative colitis
v. Grave's disease
b. Other conditions with some evidence of complement involvement:
i. Cardiovascular disease
ii. Ischaemia-reperfusion injury
iii. Traumatic brain injury
iv. Asthma
v. Multiple Sclerosis
vi. Parkinson's, Alzheimer's and Motomeurone disease all have C3d visible on histological specimens.

In particular the complement related disease may be C3 glomerulopathy, IgA nephropathy, systemic lupus erythematosus, Renal allograft nephropathy/rejection, Age related macular degeneration, Rheumatoid arthritis or Cardiovascular disease.

The sample may be any appropriate sample obtainable from a subject, from which a mutation in CFHR5 may be identified. For example, the sample may be a blood, tissue or saliva sample.

Also provided is a method for diagnosing a complement related disease, comprising identifying a mutation in the CFHR5 protein in a sample obtained from a subject.

The mutation may be any mutation, such as a shortened or lengthened form of the protein, a change in amino acid sequence, such as the deletion, insertion or substitution of one or more amino acids. Mutations due to gene fusions are also included, for example mutations in the protein brought about by recombination such that part or all of the CFHR5 gene is fused to part or all of another gene.

Mutations in the protein may be identified in a number of different ways, for example using antibodies that bind to the wild-type protein but not to the mutated protein, or vice versa or by demonstration of altered molecular weight of a species sharing immunoreactivity with the wild type protein (eg by Western Blotting). Other binding assays may be used, for example assaying for binding to purified C5, C3b, heparin or to red cells with activated C3, the mutated proteins showing a different, usually lower affinity for C5, C3b, heparin or red cells with activated C3 compared to the wild-type protein.

Also provided is the use of the wild-type CFHR5 protein, or a functional fragment thereof as a biomarker of a complement related disease, especially a complement related disease of the kidney. The inventors have found that during renal disease, CFHR5 protein is sequestered from the circulation to the kidney. Accordingly, a change in the level of CFHR5 in a subject's blood can be indicative of a complement related disease, especially of the kidney. There is provided a method of diagnosing or monitoring a complement related disease, especially of the kidney, comprising assessing the concentration of CFHR5 protein in a blood sample obtained from a subject and comparing the concentration with the expected concentration, wherein a change in concentration, especially a reduction in concentration, is indicative of the subject having a complement related disease. Alternatively, the method may comprise comparing the concentration with the concentration in a sample previously obtained from the subject, wherein a change in concentration is indicative of a change in the status, progression or severity of a complement related disease. The concentration of CFHR5 may be measured by standard techniques, such as ELISA. The term expected concentration means the concentration of CFHR5 usually found in the blood of an individual not suffering from a complement related disease. The expected concentration may be established by testing a number of such individuals and establishing a scale of normal CFHR5 concentrations. A reduction in CFHR5 concentration of at least 25%, more preferably 35%, even more preferably at least 50% is indicative of a subject having a complement related disease.

Further provided is a pharmaceutical composition comprising a protein having an amino acid sequence of the CFHR5 protein or a functional fragment thereof or a nucleic acid molecule comprising a nucleotide sequence encoding CFHR5 and a pharmaceutically acceptable carrier

Also provided is a protein having an amino acid sequence of the CFHR5 protein or a functional fragment thereof or a nucleic acid molecule comprising a nucleotide sequence encoding CFHR5 for use in therapy.

Further provided is a protein having an amino acid sequence of the CFHR5 protein or a functional fragment thereof or a nucleic acid molecule comprising a nucleotide sequence encoding CFHR5 for use in the treatment of a complement related disease.

Also provided is a method of treating a complement related disease comprising administering to a subject in need thereof CFHR5 protein, or a functional fragment thereof, such as by administering plasma, or by administering a compound that increases the CFHR5 produced by the subject. In order to treat a complement related disease it may be desirable to increase the concentration of CFHR5 in that individual's blood. This may be achieved either by administering CFHR5, especially recombinant CFHR5 to that individual. Alternatively, it may be achieved by modulating the production of CFHR5 by that individual, by for example, genetic manipulation.

Also provided is an isolated nucleic acid molecule comprising a nucleotide sequence as shown in FIG. 5.

Further provided is an isolated protein molecule encoded by the nucleic acid molecule of the invention.

The invention also provides a vector comprising the nucleic acid molecule of the invention. Any appropriate vector may be selected. The vector may be used to introduce the nucleic acid molecule into a host cell, such as bacteria, yeast, drosophila or mammalian cells. In particular, the vector may be the plasmid pCI-neo, available from Promega (Product E1841). The proteins may be expressed in COST cells.

Animals deficient in complement factor H are known from the art and exhibit complement activation, C3 depletion and complement-dependent membranoproliferative glomerulonephritis (Pickering et al Nat Gen 2002).

Other animal models are provided by the invention. Specifically provided is an animal model of a complement related disease, comprising an animal having a mutation in the CFHR5 gene. Also provided is a transgenic animal which expresses wildtype or mutant human CFHR5. Expression of the CFHR5 may be under the control of an inducible promoter. Also provided are animals having the following genotypes CFH-; CFH-/CFHR5WT; CFH-/CFHR5mut), produced by crossing the transgenic animals of the invention with CFH deficient animals. Such animals may be used to compare the severity of disease in the varying genotypes. The transgenic animal of the invention is preferably a non-human mammal, especially a rodent, such as a mouse. The animal may also be a larger mammal, especially a mammal that could be used for xenotransplantation, such as a pig.

There is provided a method for identifying compounds that may be useful in the treatment of complement related disorders comprising administering to the animal model a candidate compound and observing the animal for a change in clinical signs.

Also provided are compounds identified by the methods, especially for use in therapy.

### BRIEF DESCRIPTION OF THE DRAWINGS

The invention will now be described in detail by way of example only, with reference to the figures:

FIG. 1. Familial C3 glomerulonephritis

(A) Pedigrees of two ostensibly unrelated families from the Troodos Mountains. Haplotypes at the region of maximum linkage on chromosome 1 are shown. A haplotype (boxed, HapMap coordinates 190809281 to 199492315) cosegregates with renal disease in both families and includes the Regulators of Complement Activation gene cluster.

(B) First renal biopsy from V-4 (family one). (i) Light microscopic appearance of glomerulus showing a small area of mesangial hypercellularity (arrow). (ii) Immunoperoxidase for C3 showing granular staining on the capillary wall. (iii) and (iv) Electron micrographs showing prominent mesangial (arrow, iii), and sub-endothelial (arrows, iv) with occasional sub-epithelial electron-dense deposits (white arrow, iii).

(C) Genetic linkage study. Using an autosomal dominant model and scoring all those with hematuria as affected, a LOD score of 3.40 at a locus on chromosome 1 was detected, the peak of which contains the shared haplotype and the Regulators of Complement Activity (RCA) gene cluster.

FIG. 2. Heterozygous internal duplication of complement factor H-related protein 5 (CFHR5).

(A) Multiple Ligation Probe Assay (MLPA) analysis across the CFH and CFHR genes using genomic DNA from individuals from family one (IV-6 and V-4) and family two (II-1, II-2 and III-2). This demonstrated the presence of 3 copies of exons two and three of CFHR5 in affected individuals from family one (IV-6 and V4) and family two (II-2 and III-2) but not in the unaffected individual in family two (II-1). The analysed members of family 2 but not family 1 were also noted to have heterozygous deletion of CFHR1 and 3, a common polymorphism.¹⁶

(B) Confirmation of CFHR5 internal duplication. Southern blot of EcoR1-digested genomic DNA from unrelated control individuals (C1 and C2) and 5 individuals from both families was probed with exon 2 of CFHR5. This demonstrated the presence of an additional 6.3 kb fragment in affected individuals from family 1 (IV-6 and V-4) and family 2 (II-2 and III-2). This fragment was not present in the two unrelated control individuals or in an unaffected individual from family 2 (II-1) where only the expected 7.9 kb band was seen. Using primers positioned in CFHR5 exon 2 and intron 3 (arrows) no amplification occurs from wild-type sequence but internal duplication of exons 2 and 3 leads to a product of 4.8 kb. Sequencing of the product confirmed the position and size of the duplication. E=EcoRI, numbered black rectangles denote CFHR5 exons, EcoRI restriction fragments indicated by double-headed arrows and duplicated region indicated in red.

(C) PCR was performed with three primers designed to give a single product of 298 by in the wild-type allele (arrowhead) and a 222 by product in the allele associated with CFHR5 internal duplication (arrow). The 222 by product was seen in affected but not unaffected members of both families.

FIG. 3. Detection and functional analysis of CFHR5 variant protein.

(A) Detection of CFHR5 in serum. Western blotting of serum using a polyclonal anti-CFHR5 antibody demonstrated an aberrant band in individuals with genetic evidence of internal duplication of CFHR5 exons 2 and 3. Duplication of exons 2 and 3 would be predicted to generate a CFHR5 protein containing two additional SCR domains (schematic right panel and denoted by CFHR5¹²¹²³⁻⁹) since SCR domains 1 and 2 of CFHR5 are encoded by exons 2 and 3 respectively. The initial amino acid sequence of SCR1 is EGTLCD with the initial glutamic acid (E) encoded by exon one. Since the duplicated SCR1 (orange shaded domain) is encoded only by exon 2 the predicted SCR sequence lacks the initial glutamic acid. Splicing of exon 3 upstream of the exon 2 sequence results in a change in the initial codon of the duplicated SCR1 so that the second amino acid in the EGTL sequence is arginine (R) not glycine (G). Hence, the initial amino acid sequence of the duplicated SCR1 is predicted to be RTLCD. The remaining duplicated SCR1 amino acid sequence and the entire duplicated SCR2 amino acid sequence is identical to wild-type.

(B) Functional analysis of CFHR5 proteins. CFHR5 has previously been shown to bind to erythrocytes lysed by complement.¹⁷ Patient serum was incubated with chicken erythrocytes which spontaneously activate the AP resulting in erythrocyte lysis and binding of CFHR5 on the disrupted membranes. The relative amounts of unbound CFHR5 proteins (supernatant) and bound (erythrocyte membrane pellet) was determined by western blotting (left panel). Binding of the CFHR5¹²¹²³⁻⁹ variant to complement-lysed erythrocyte membranes was markedly impaired with the majority remaining in the unbound supernatant fraction (lane 4). This was a consistent finding over three experiments (right panel).

(C) Schematic showing putative role of CFHR5 in C3 glomerulopathy. Under physiological conditions plasma CFH regulates the AP efficiently in plasma whilst CFHR5 removes C3 fragments that reach the glomerular basement membrane (GBM) preventing abnormal GBM C3 accumulation. This homeostatic mechanism can be perturbed by either dysregulation of plasma AP activation (central panel, examples include CFH deficiency or C3NeF) or by defective processing of GBM C3 by CFHR5 (right panel). In either circumstance C3 accumulates triggering C3 glomerulopathy.

FIG. 4 shows the nucleotide sequence of the wild-type CFHR5 gene.

FIG. 5 shows the nucleotide sequence of a mutated CFHR5 gene.

### DETAILED DESCRIPTION OF THE INVENTION

### Patients and Clinical Details

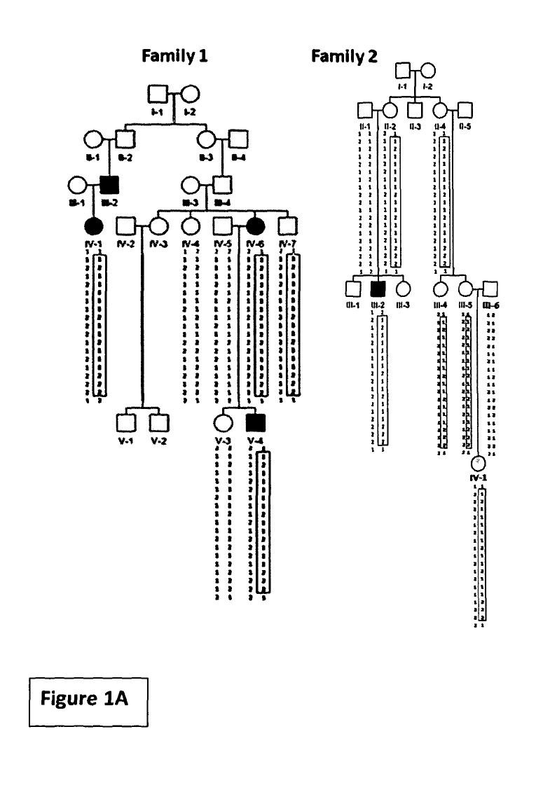

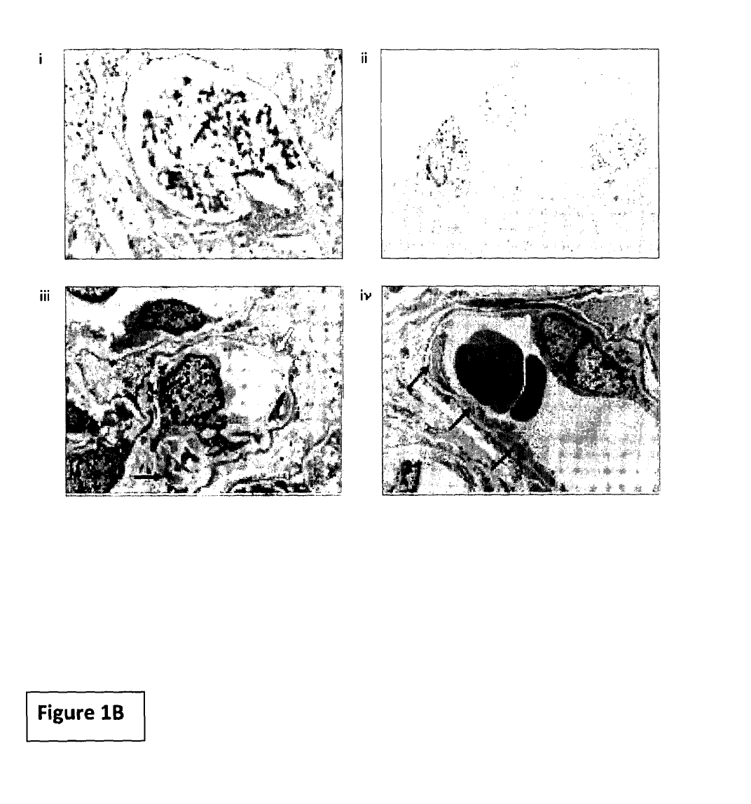

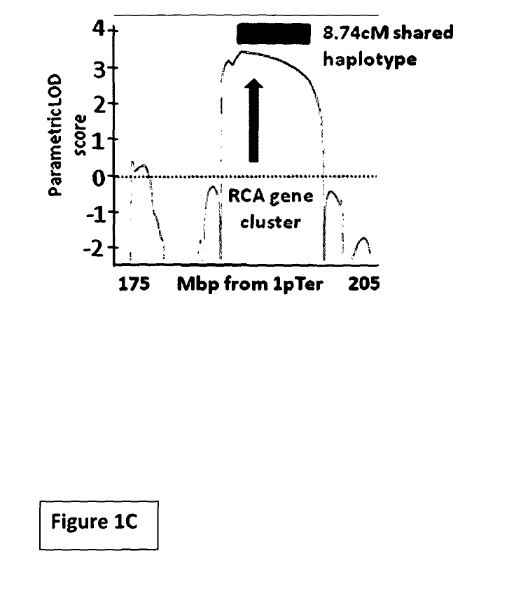

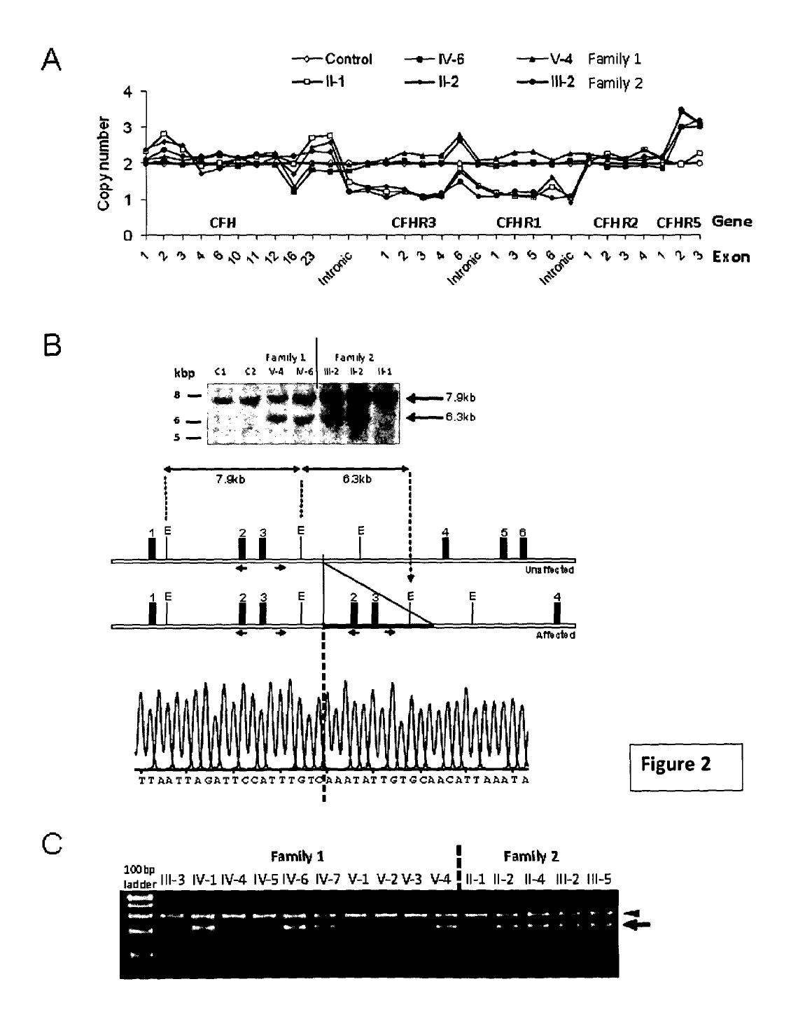

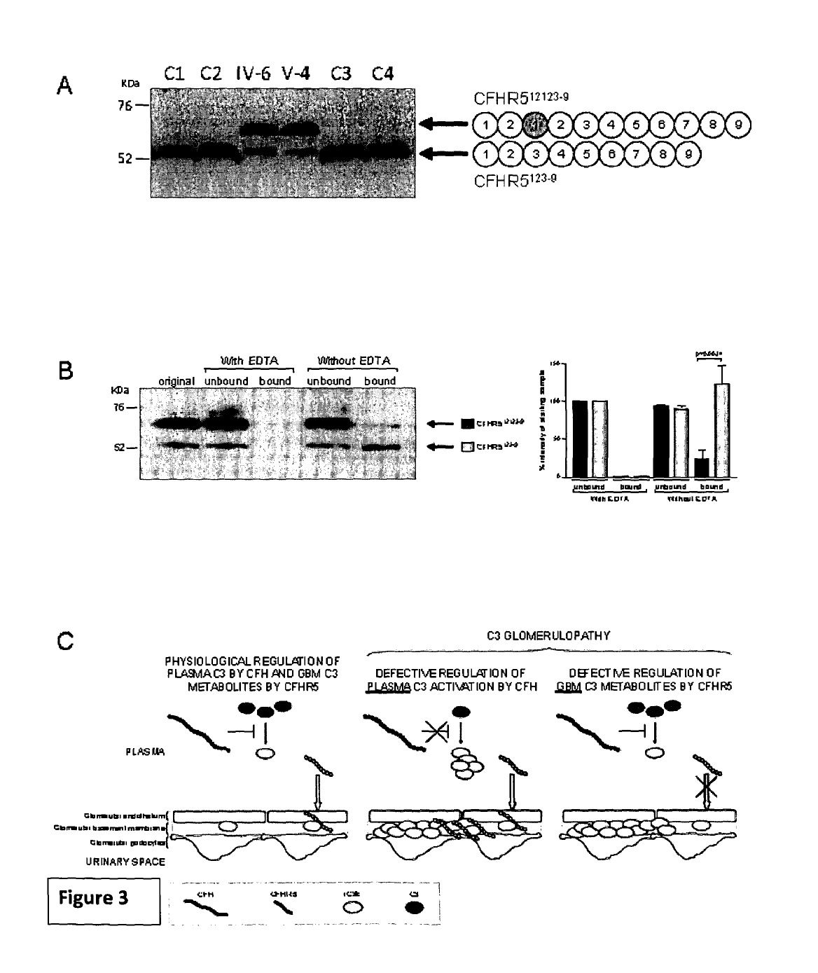

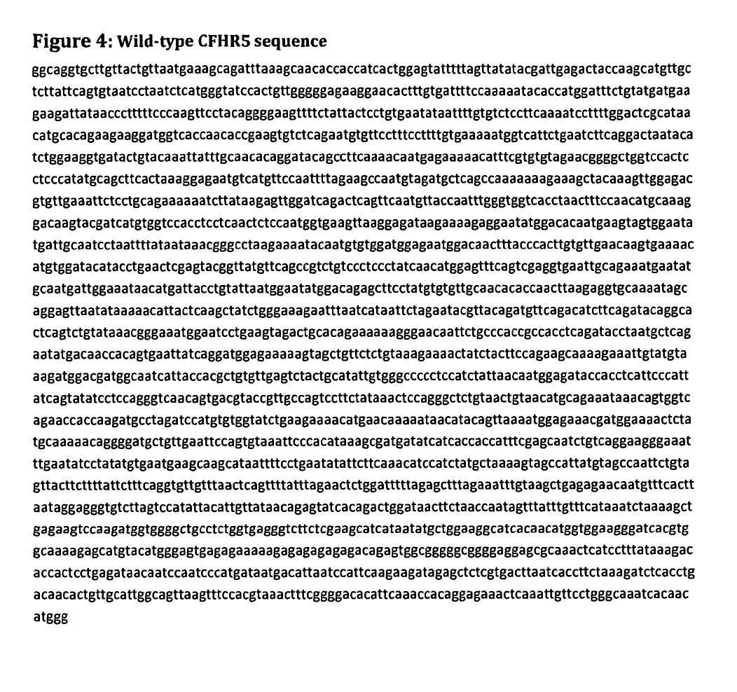

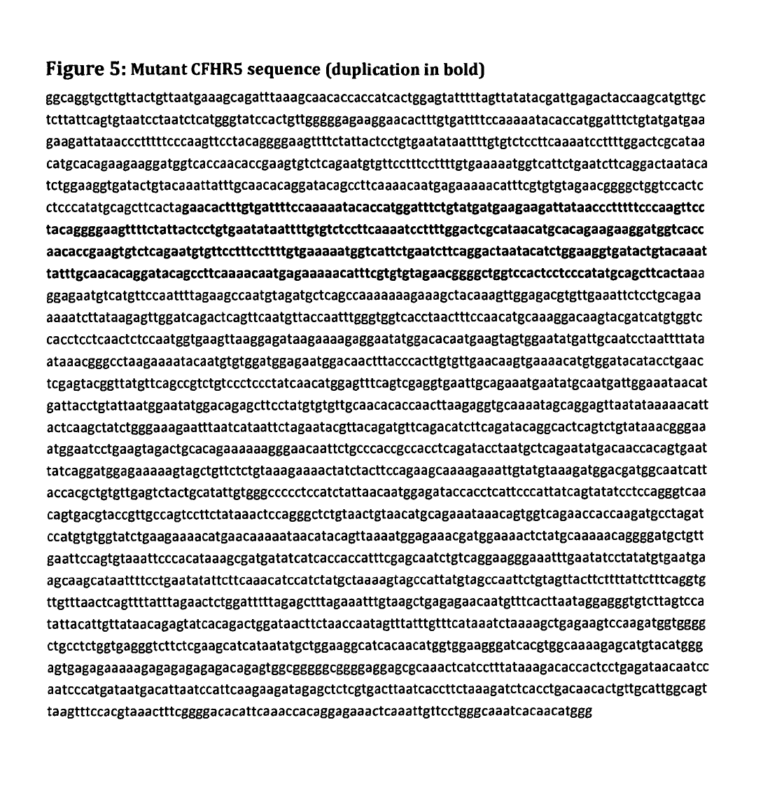

The study was approved by the Local Research Ethics Committees. Family 1. The index case (V-4 in FIG. 1a) presented aged 17 with hypertension, microscopic hematuria and episodic macroscopic hematuria coinciding with upper respiratory tract infections. Renal biopsy demonstrated mild expansion of the mesangial matrix and increased glomerular cellularity, segmental capillary wall thickening and focal tubular atrophy. Electron microscopy showed subendothelial and mesangial electron dense deposits with infrequent subepithelial deposits. There was positive staining for complement C3 but not for immunoglobulins or C1q in these areas (FIG. 1b). Serum complement C3 and C4 levels were normal (C3 1.05 g/l, normal range 0.7-1.7 and C4 0.24 g/l, normal range 0.16-0.54) and there was no clinical or serological evidence of chronic infection, C3 nephritic factor or microangiopathy. Over the next 10 years proteinuria (<1 g per day) and renal impairment developed, with a fall in 51Cr-EDTA clearance from >100 ml/min at original presentation to 70 ml/min at age 26. A repeat biopsy showed increased tubular atrophy and glomerular obsolescence. There was a family history of renal disease and, where performed, findings on biopsy were essentially identical (FIG. 1a). Retinal photography in individuals V-4 and IV-6 was normal with no evidence of drusen and circulating factor H levels were normal (548 and 631 mg/l respectively). Family 2. An ostensibly unrelated individual (III-2 in FIG. 1a) presented aged 38 with microscopic hematuria, episodic macroscopic hematuria coincident with upper respiratory tract infections, proteinuria (0.5-1 g per day), hypertension, renal impairment (serum creatinine 1.8 mg/dl) and a family history of renal disease (FIG. 1a). Renal biopsy demonstrated C3 glomerulonephritis. Both families originated from the same valley of the Troodos mountains in Cyprus.

### Materials and Methods

Genome-wide linkage study. DNA was extracted from blood or saliva (Oragene, DNA Genotek, Canada). Genotypes and haplotypes of 6008 single nucleotide polymorphisms (SNPs) (Linkage IV panel, Illumina, Calif.) in the families were analyzed using EasyLINKAGE,¹⁸ PEDCHECK,¹⁹ GENEHUNTERv2.1²⁰ and HAPLOPAINTER.²¹ Bidirectional sequencing of the exons of candidate genes was performed following polymerase chain reaction (PCR) amplification (primers available on request). CFHR5 internal duplication. Multiplex Ligation-Dependent Probe Amplification (MLPA) was performed on unamplified genomic DNA using the P236 A1 ARMD mix 1 from MRC-Holland (Amsterdam, The Netherlands). Full details are available at www.mrc-holland.com). Southern blotting was performed on genomic DNA digested with EcoRI (New England Biolabs, MA). Membranes were probed with a ³²P-labelled 371 base pair sequence containing exon 2 of CFHR5. PCR amplification of the CFHR5 duplication insertion point used primers: 5′-TGGAAGCCTGTGGTATAAATGA-3′ and 5′-TCCGGCACATCCTTCTCTAT-3′ (FIG. 2b). PCR amplifying both CFHR5 alleles in a single reaction used primers 5′-GATTCCATTTGTCAAATATTG-3′, 5′- TCTTCTCCAAAACTATCTAATGTCAA-3′ and 5′-TTTGAATGCTGTTTTAGCTCG-3′. Detection of CFHR5 in sera and functional analysis. CFHR5 was detected by the Western blotting of serum using rabbit antiserum¹⁷ (a gift from J. McRae, Immunology Research Centre, Melbourne, Australia). Wild-type and mutant CFHR5 functional tests were performed with washed chicken erythrocytes (CE) suspended 1:10 (v/v) in 25 μl saline and incubated at 37° C. overnight with 50 μl of serum and 425 μl of saline, with or without 10 mM EDTA to inhibit complement activation.¹⁷ After centrifugation at 13000 rpm CFHR5 in the fractions was assayed by Western blotting. Binding of CFHR5 to heparin was assayed as previously described.²²

### Results

In view of the previous association of mutations in AP components with C3GN, we sequenced the exons of the genes Bf, C3, CFH, CFI, CD46 and the CFH-related genes CFHR1 and CFHR5 in individuals V-4 and IV-6 from family 1. No coding or splice site mutations were detected in these genes. A genome wide SNP-based analysis established linkage in family 1 to an 18 cM region of chromosome 1q31-32 (LOD 2.22), which contains the genes for CFH and CFHR1-5 (termed the Regulators of Complement Activation (RCA) cluster). With the addition of family 2 (scoring female relatives with microscopic hematuria as affected) the combined LOD score at this locus was 3.40 (FIG. 1c). In addition, a haplotype comprising 15 SNPs and spanning 8.74cM within the linked region and including the RCA cluster was shared between all affected members of both families (FIG. 1a), consistent with inheritance of an allele at this locus from a single common ancestor. Other loci containing complement genes were excluded (LOD<-2). MLPA analysis in V-4 (C3GN) and IV-6 (C3GN) from family 1 and III-2 (C3GN), II-2 (microscopic hematuria) and II-1 (unaffected) from family 2 showed heterozygosity for a duplication of exons 2 and 3 of CFHR5 in affected individuals but not the unaffected family member (FIG. 2a). In addition, deletion of the CFHR1 and CFHR3 genes was noted in all 3 members of family 2 tested but was not observed in either member of family 1 (FIG. 2a).

Southern blot of genomic DNA probed with CFHR5 exon 2 revealed an additional 6.3 kbp band in individuals with renal disease and the boundary of the duplication was identified by resequencing a PCR product (FIG. 2b). A PCR reaction demonstrated the presence of the internal duplication in all individuals in both families with C3GN and in obligate carriers (FIG. 2c). One individual (V-2 in family 1) was noted to have microscopic hematuria on a single occasion but did not have the duplication. To exclude the possibility that this allele is a normal variant we showed that it was not present in 102 unrelated individuals from the UK 1958 birth cohort. To explore its possible frequency in Cyprus we screened DNA from individuals collected as control subjects in the MASTOS study²³. Heterozygosity for the mutation was identified in a single individual amongst the 1015 that we screened, implying that this is a rare allele in the Cypriot population. The ethical approval under which this investigation was performed did not allow demographic or clinical details to be obtained, so we do not know if the individual has associated clinical manifestations.

In order to identify whether any additional affected individuals exist in Cyprus we screened a cohort of 84 Greek-Cypriot patients with sporadic renal disease for the presence of the mutation and identified 3 males and one female patient with the mutation (4.8%). None of these individuals reported ancestry in the Troodos mountains. A histological diagnosis was not available in 41 of these (of which one male and one female were affected). The diagnoses for the remaining 43 patients are recorded in table 1. In addition, 2 further separate families in whom microscopic haematuria segregated as an autosomal dominant trait and direct exon sequencing had excluded known mutations in the genes COL4A3-COL4A4²⁴ were investigated. Both families comprised 5 affected individuals, all of whom carried the CFHR5 duplication which was not identified in any unaffected relatives. A renal biopsy had been performed in a single individual from one of these families and this showed C3GN. Neither of these families could trace their ancestry to the Troodos mountains. We conclude that this CFHR5 duplication accounts for a significant proportion of renal disease in Cyprus and is not confined to the Troodos region of the island.

CFHR5 consists of nine short consensus repeat (SCR) domains (CFHR5¹²³⁻⁹, superscript numbers denoting SCRs). Duplication of exons 2 and 3 predicts a CFHR5 protein containing two additional SCRs (denoted by CFHR5¹²¹²³⁻⁹) since SCRs 1 and 2 of CFHR5 are encoded by exons 2 and 3 respectively (FIG. 3a). Western blot of sera from affected individuals demonstrated, in addition to the normal CFHR5 protein, the presence of a slower migrating protein, consistent with the predicted molecular weight of the CFHR5¹²¹²³⁻⁹ protein (FIG. 3a). Functional assays demonstrated that the binding of the CFHR5¹²¹²³⁻⁹ in patient sera to complement-lysed chick erythrocytes was markedly reduced compared to CFHR5¹²³⁻⁹ (FIG. 3b). Binding of both CFHR5 proteins to erythrocyte surfaces was inhibited by 10 mM EDTA indicating that it was dependent on complement activation. Furthermore, heparin affinity chromatography demonstrated that the CFHR5¹²¹²³⁻⁹ protein had reduced affinity for heparin since it was eluted at a lower concentration (300 mM NaCl) compared to CFHR5¹²³⁻⁹ (which eluted at 350 mM, data not shown). Taken together these data show inheritance of a defective CFHR5 allele which causes C3GN.

### DISCUSSION

CFHR5 is a member of the CFH gene family which comprises CFH and the 5 CFH related genes (CFHR1-5). The in vivo importance of CFH as the major regulator of plasma AP activity is illustrated by the severe AP dysregulation, resulting in secondary depletion of plasma C3, which occurs in individuals with complete genetic CFH deficiency.²⁵ CFH also appears to play a physiological role in protecting renal endothelium since mutations that impair binding to this surface predispose to aHUS.⁸ Recently the association of genetic variants in CFHR genes with disease suggested they are also important. Thus deletion of CFHR1 and CFHR3 genes is a common polymorphism that appears to confer altered susceptibility to AMD and aHUS (reviewed in⁸) and certain common CFHR5 variants preferentially associate with aHUS²⁶ and DDD.²⁷ CFHR5 is unique among CFHR proteins in having detectable complement regulatory activity in vitro.^{28,29} Furthermore, in vitro, CFHR5 can bind to heparin and to activated C3, functions that would enable interaction with complement deposited in tissues.²⁸ Consistent with this, CFHR5 has been detected in kidney when there is complement deposition.26 CFHR5 is a 65 kDa protein with 9 SCR domains¹⁷; the internal duplication we identified adds an additional two SCR domains. Our functional studies tested the ability of the CFHR5 ¹²¹²³⁻⁹ protein to mediate two of the known functions of the normal CFHR5 ¹²³⁻⁹ protein: the ability to bind to complement on a surface and heparin binding. In both instances the CFHR5¹²¹²³⁻⁹ protein demonstrated reduced affinity compared with normal CFHR5¹²³⁻⁹ protein, suggesting that the CFHR5¹²¹²³⁻⁹ protein is a ‘loss of function’ variant although we do not exclude that it may also exert a dominant negative effect. Western blot analysis of serum from individuals with the CFHR5¹²¹²³⁻⁹ protein variant showed that the intensity of the normal CFHR5 ¹²³⁻⁹ protein band was reduced compared to control samples. In part this is likely to be a dose effect, reflecting heterozygosity for the wild type CFHR5¹²³⁻⁹ allele but would also be consistent with greater sequestration of wild type CFHR5 protein by complement deposits within the C3GN lesions. The fact that this allele was identified in 2 ostensibly unrelated families (in whom it can be inferred from the size of their shared haplotype that the most recent common ancestor at the locus in question was most likely to have lived approximately 10 generations ago³⁰) suggested that a larger population of affected individuals existed. This supposition was supported firstly by the detection of the allele in an individual in a large, independently obtained sample of the Cypriot population and secondly by the identification of multiple additional affected individuals and families among Cypriot patients with renal disease. The high penetrance of haematuria and wide geographical distribution of the ancestry within Cyprus of these individuals and families suggests that this disease may account for a significant proportion of renal disease affecting inhabitants of the island and their descendants across the world.

The current study provides a molecular explanation for these cases of C3GN. We did not detect this specific CFHR5 mutation in 36 other sporadic cases from France and the UK (data not shown). Although mutations in other complement regulatory genes and/or the presence of C3NeF have been identified in C3GN, in the majority of cases no molecular cause has yet been identified.¹³ Our data suggest that other genetic mechanisms impairing CFHR5 function may be present in these individuals.

In summary, we describe a novel genetic cause for C3GN which is endemic in Cyprus and provide direct evidence that CFHR5 plays an important physiological role in the processing of complement within the kidney (FIG. 3c).

### FURTHER EXAMPLES

The inventors have identified the CFHR5 mutation in over 100 people from across Cyprus, 90% of whom have evidence of kidney disease. They have noted that the disease recurs following a transplant. This proves that the disease results from a circulating abnormality and thus implies that therapeutic targeting of the circulation to increase the level of functional CFHR5 is likely to be of benefit. Plasma exchange resulted in immediate cessation of macroscopic haematuria and reduction in serum creatinine. This was associated with a relative reduction in levels of circulating mutant protein (assessed by Western Blotting) and suggests that manipulating activity of complement in the circulation may be of therapeutic benefit.

### REFERENCES

1. Zarkadis I K, Mastellos D, Lambris J D: Phylogenetic aspects of the complement system. Dev Comp Immunol 2001; 25: 745-762.
2. Nonaka M, Azumi K, Ji X et al: Opsonic complement component C3 in the solitary ascidian, Halocynthia roretzi. J Immunol 1999; 162: 387-391.
3. Walport M J: Complement. First of two parts. N Engl J Med 2001; 344: 1058-1066.
4. Pickering M C, Cook H T: Translational mini-review series on complement factor H: renal diseases associated with complement factor H: novel insights from humans and animals. Clin Exp Immunol 2008; 151: 210-230.
5. Koeijvoets K C, Mooijaart S P, Dallinga-Thie G M et al: Complement factor H Y402H decreases cardiovascular disease risk in patients with familial hypercholesterolaemia. Eur Heart J2009; 30: 618-623.
6. Volcik K A, Ballantyne C M, Braun M C, Coresh J, Mosley T H, Boerwinkle E: Association of the complement factor H Y402H polymorphism with cardiovascular disease is dependent upon hypertension status: The ARIC study. Am J Hypertens 2008; 21: 533-538.
7. Mooijaart S P, Koeijvoets K M, Sijbrands E J, Daha M R, Westendorp R G: Complement Factor H polymorphism Y402H associates with inflammation, visual acuity, and cardiovascular mortality in the elderly population at large. Exp Gerontol 2007; 42: 1116-1122.
8. de Cordoba S R, de Jorge E G: Translational mini-review series on complement factor H: genetics and disease associations of human complement factor H. Clin Exp Immunol 2008; 151: 1-13.
9. Fakhouri F, Jablonski M, Lepercq J et al: Factor H, membrane cofactor protein, and factor I mutations in patients with hemolysis, elevated liver enzymes, and low platelet count syndrome. Blood 2008; 112: 4542-4545.
10. Goicoechea de Jorge E, Harris C L, Esparza-Gordillo J et al: Gain-of-function mutations in complement factor B are associated with atypical hemolytic uremic syndrome. Proc Natl Acad Sci USA 2007; 104: 240-245.
11. Fremeaux-Bacchi V, Miller E C, Liszewski M K et al: Mutations in complement C3 predispose to development of atypical hemolytic uremic syndrome. Blood 2008; 112: 4948-4952.
12. Smith R J, Alexander J, Barlow P N et al: New approaches to the treatment of dense deposit disease. J Am Soc Nephrol 2007; 18: 2447-2456.
13. Servais A, Fremeaux-Bacchi V, Lequintrec M et al: Primary glomerulonephritis with isolated C3 deposits: a new entity which shares common genetic risk factors with haemolytic uraemic syndrome. J Med Genet 2007; 44: 193-199.
14. Meri S, Koistinen V, Miettinen A, Tornroth T, Seppala I J: Activation of the alternative pathway of complement by monoclonal lambda light chains in membranoproliferative glomerulonephritis. J Exp Med 1992; 175: 939-950.
15. Dragon-Durey M A, Loirat C, Cloarec S et al: Anti-Factor H autoantibodies associated with atypical hemolytic uremic syndrome. J Am Soc Nephrol 2005; 16: 555-563.
16. Zipfel P F, Edey M, Heinen S et al: Deletion of complement factor H-related genes CFHR1 and CFHR3 is associated with atypical hemolytic uremic syndrome. PLoS Genet 2007; 3: e41.
17. McRae J L, Cowan P J, Power D A et al: Human factor H-related protein 5 (FHR-5). A new complement-associated protein. J Biol Chem 2001; 276: 6747-6754.
18. Hoffmann K, Lindner T H: easyLINKAGE-Plus—automated linkage analyses using large-scale SNP data. Bioinformatics 2005; 21: 3565-3567.
19. O'Connell J R, Weeks D E: PedCheck: a program for identification of genotype incompatibilities in linkage analysis. Am J Hum Genet 1998; 63: 259-266.
20. Markianos K, Daly M J, Kruglyak L: Efficient multipoint linkage analysis through reduction of inheritance space. Am J Hum Genet 2001; 68: 963-977.
21. Thiele H, Nurnberg P: HaploPainter: a tool for drawing pedigrees with complex haplotypes. Bioinformatics 2005; 21: 1730-1732.
22. Pickering M C, de Jorge E G, Martinez-Barricarte R et al: Spontaneous hemolytic uremic syndrome triggered by complement factor H lacking surface recognition domains. J Exp Med 2007; 204: 1249-1256.
23. Loizidou M A, Michael T, Neuhausen S L et al: Genetic polymorphisms in the DNA repair genes XRCC1, XRCC2 and XRCC3 and risk of breast cancer in Cyprus. Breast Cancer Res Treat 2008; 112: 575-579.
24. Voskarides K, Damianou L, Neocleous V et al: COL4A3/COL4A4 mutations producing focal segmental glomerulosclerosis and renal failure in thin basement membrane nephropathy. J Am Soc Nephrol 2007; 18: 3004-3016.
25. Thompson R A, Winterborn M H: Hypocomplementaemia due to a genetic deficiency of beta 1H globulin. Clin Exp Immunol 1981; 46: 110-119.
26. Monteferrante G, Brioschi S, Caprioli J et al: Genetic analysis of the complement factor H related 5 gene in haemolytic uraemic syndrome. Mol Immunol 2007; 44: 1704-1708.
27. Abrera-Abeleda M A, Nishimura C, Smith J L et al: Variations in the complement regulatory genes factor H (CFH) and factor H related 5 (CFHR5) are associated with membranoproliferative glomerulonephritis type II (dense deposit disease). J Med Genet 2006; 43: 582-589.
28. Murphy B, Georgiou T, Machet D, Hill P, McRae J: Factor H-related protein-5: a novel component of human glomerular immune deposits. Am J Kidney Dis 2002; 39: 24-27.
29. McRae J L, Duthy T G, Griggs K M et al: Human factor H-related protein 5 has cofactor activity, inhibits C3 convertase activity, binds heparin and C-reactive protein, and associates with lipoprotein. J Immunol 2005; 174: 6250-6256.
30. Genin E, Tullio-Pelet A, Begeot F, Lyonnet S, Abel L: Estimating the age of rare disease mutations: the example of Triple-A syndrome. J Med Genet 2004; 41: 445-449.

1. A method for diagnosing a complement related disease, comprising:
identifying a mutation in CFHR5 gene in a sample obtained from a subject.

2. The method of claim 1, wherein the complement related disease is selected from the group consisting of:
a. C3 glomerulopathy;
b. Haemolytic uraemic syndrome;
c. Dense deposit disease (also known as MPGN Type II or C3Neph);
d. IgA nephropathy;
e. Mesangiocapillary (membranoproliferative) glomerulonephritis (MPGN);
f. systemic lupus erythematosus;
g. antiphospholipid syndrome;
h. Renal allograft nephropathy/rejection;
i. ANCA associated vasculitis;
j. Age related macular degeneration;
k. Thrombotic thrombocytopaenic purpura (TTP);
l. HELLP syndrome of pregnancy;
m. Paroxysmal Nocturnal Haemoglobinuria;
n. Rheumatoid arthritis;
o. Myositis/myocarditis;
p. Pemphigoid/pemphigus/epidermolysis bullosa;
q. Crohn's disease/ulcerative colitis;
r. Grave's diseases
s. Cardiovascular diseases
t. Ischaemia-reperfusion injury;
u. Traumatic brain injury;
.Asthma;
w. Multiple Sclerosis;
x. Parkinson's disease;
y. Alzheimer's disease; and
z. Motomeurone disease.

3. The method of claim 2, wherein the complement related disease is selected from the group consisting of C3 glomerulopathy, 1 gA nephropathy, systemic lupus erythematosus, renal allograft nephropathy/rejection, age related macular degeneration, rheumatoid arthritis and cardiovascular disease.

4. A method for diagnosing a complement related disease, comprising identifying a mutation in CFHR5 protein in a sample obtained from a subject.

5. A pharmaceutical composition comprising a protein having the amino acid sequence of CFHR5 protein or a functional fragment thereof, or a nucleic acid molecule comprising a nucleotide sequence encoding CFHR5 and a pharmaceutically acceptable carrier.

6. A protein having the amino acid sequence of CFHR5 protein or a functional fragment thereof, or a nucleic acid molecule comprising a nucleotide sequence encoding CFHR5.

7. (canceled)

8. An isolated nucleic acid molecule comprising a nucleotide sequence as shown in FIG. 5.

9. An isolated protein molecule encoded by the nucleic acid molecule of claim 8.

10. A vector comprising the nucleic acid molecule claim 8.

11. A host cell comprising the vector of claim 10.

12. A non-human animal model of a complement related disease, comprising: an animal having a mutation in the CFHR5 gene.

13. A transgenic, non-human animal which expresses wild-type or mutant human CFHR5.

14. A transgenic, non-human animal which has a gentotype selected from the group consisting of CFH⁻; CFH-/CFHR5WT; and CFH-/CFHR5mut).

15. A method for identifying compounds that may be useful in the treatment of complement related disorders comprising: administering to an animal according to any of claims 12 to 14 a candidate compound and observing the animal for a change in clinical signs.

16. (canceled)

17. A method of diagnosing or monitoring a complement related disease, comprising; assessing the concentration of CFHR5 protein in a blood sample obtained from a subject and comparing the concentration with the expected concentration, wherein a change in concentration is indicative of that the subject has a complement related disease.

18. A method of diagnosing or monitoring a complement related disease, comprising: assessing the concentration of CFHR5 protein in a blood sample obtained from a subject and comparing the concentration with the concentration in a sample previously obtained from the subject, wherein a change in concentration is indicative of a change in status of the complement related disease in the subject, progression or severity of a complement related disease.

19. A method of treating a complement related disease comprising: administering to a subject in need thereof CFHR5 protein, or a functional fragment thereof, or a compound that increases the CFHR5 produced by the individual.

20. The method of claim 1 or 2, wherein the complement related disease is a renal disorder caused by complement dysregulation.

21. The method of claim 1 or 2, wherein the complement related disease is a renal disease in which complement dysregulation is a consequence of antibody activation.

22. The method of claim 1, wherein the complement related disease is MPGN associated with Hepatitis B.

23. The method of claim 1, wherein the complement related disease is MPGN associated with Hepatitis C.

24. The method of claim 1, wherein the complement related disease is MPGN associated with a chronic bacterial infection.

25. The method of claim 1 wherein the complement related disease is MPGN associated with tuberculosis.

26. The method of claim 1 wherein the complement related disease is MPGN associated with a chronic infected ulcer or abscess.

27. The method of claim 1 or 2, wherein the complement related disease is an autoimmune disorder.

28. The method of claim 1 or 2, wherein histological specimens of the complement related disease display C3d.


SEQUENCE LISTING (2 sequences)

SEQ ID NO: 1 - DNA, 2481 bp, Homo sapiens
ggcaggtgcttgttactgttaatgaaagcagatttaaagcaacaccaccatcactggagtatttttagttatatacgattgagactaccaagcatgttgctcttattcagtgtaatcctaatctcatgggtatccactgttgggggagaaggaacactttgtgattttccaaaaatacaccatggatttctgtatgatgaagaagattataaccctttttcccaagttcctacaggggaagttttctattactcctgtgaatataattttgtgtctccttcaaaatccttttggactcgcataacatgcacagaagaaggatggtcaccaacaccgaagtgtctcagaatgtgttcctttccttttgtgaaaaatggtcattctgaatcttcaggactaatacatctggaaggtgatactgtacaaattatttgcaacacaggatacagccttcaaaacaatgagaaaaacatttcgtgtgtagaacggggctggtccactcctcccatatgcagcttcactaaaggagaatgtcatgttccaattttagaagccaatgtagatgctcagccaaaaaaagaaagctacaaagttggagacgtgttgaaattctcctgcagaaaaaatcttataagagttggatcagactcagttcaatgttaccaatttgggtggtcacctaactttccaacatgcaaaggacaagtacgatcatgtggtccacctcctcaactctccaatggtgaagttaaggagataagaaaagaggaatatggacacaatgaagtagtggaatatgattgcaatcctaattttataataaacgggcctaagaaaatacaatgtgtggatggagaatggacaactttacccacttgtgttgaacaagtgaaaacatgtggatacatacctgaactcgagtacggttatgttcagccgtctgtccctccctatcaacatggagtttcagtcgaggtgaattgcagaaatgaatatgcaatgattggaaataacatgattacctgtattaatggaatatggacagagcttcctatgtgtgttgcaacacaccaacttaagaggtgcaaaatagcaggagttaatataaaaacattactcaagctatctgggaaagaatttaatcataattctagaatacgttacagatgttcagacatcttcagatacaggcactcagtctgtataaacgggaaatggaatcctgaagtagactgcacagaaaaaagggaacaattctgcccaccgccacctcagatacctaatgctcagaatatgacaaccacagtgaattatcaggatggagaaaaagtagctgttctctgtaaagaaaactatctacttccagaagcaaaagaaattgtatgtaaagatggacgatggcaatcattaccacgctgtgttgagtctactgcatattgtgggccccctccatctattaacaatggagataccacctcattcccattatcagtatatcctccagggtcaacagtgacgtaccgttgccagtccttctataaactccagggctctgtaactgtaacatgcagaaataaacagtggtcagaaccaccaagatgcctagatccatgtgtggtatctgaagaaaacatgaacaaaaataacatacagttaaaatggagaaacgatggaaaactctatgcaaaaacaggggatgctgttgaattccagtgtaaattcccacataaagcgatgatatcatcaccaccatttcgagcaatctgtcaggaagggaaatttgaatatcctatatgtgaatgaagcaagcataattttcctgaatatattcttcaaacatccatctatgctaaaagtagccattatgtagccaattctgtagttacttcttttattctttcaggtgttgtttaactcagttttatttagaactctggatttttagagctttagaaatttgtaagctgagagaacaatgtttcacttaataggagggtgtcttagtccatattacattgttataacagagtatcacagactggataacttctaaccaatagtttatttgtttcataaatctaaaagctgagaagtccaagatggtggggctgcctctggtgagggtcttctcgaagcatcataatatgctggaaggcatcacaacatggtggaagggatcacgtggcaaaagagcatgtacatgggagtgagagaaaaagagagagagagacagagtggcgggggcggggaggagcgcaaactcatcctttataaagacaccactcctgagataacaatccaatcccatgataatgacattaatccattcaagaagatagagctctcgtgacttaatcaccttctaaagatctcacctgacaacactgttgcattggcagttaagtttccacgtaaactttcggggacacattcaaaccacaggagaaactcaaattgttcctgggcaaatcacaacatggg

SEQ ID NO: 2 - DNA, 2853 bp, Homo sapiens
ggcaggtgcttgttactgttaatgaaagcagatttaaagcaacaccaccatcactggagtatttttagttatatacgattgagactaccaagcatgttgctcttattcagtgtaatcctaatctcatgggtatccactgttgggggagaaggaacactttgtgattttccaaaaatacaccatggatttctgtatgatgaagaagattataaccctttttcccaagttcctacaggggaagttttctattactcctgtgaatataattttgtgtctccttcaaaatccttttggactcgcataacatgcacagaagaaggatggtcaccaacaccgaagtgtctcagaatgtgttcctttccttttgtgaaaaatggtcattctgaatcttcaggactaatacatctggaaggtgatactgtacaaattatttgcaacacaggatacagccttcaaaacaatgagaaaaacatttcgtgtgtagaacggggctggtccactcctcccatatgcagcttcactagaacactttgtgattttccaaaaatacaccatggatttctgtatgatgaagaagattataaccctttttcccaagttcctacaggggaagttttctattactcctgtgaatataattttgtgtctccttcaaaatccttttggactcgcataacatgcacagaagaaggatggtcaccaacaccgaagtgtctcagaatgtgttcctttccttttgtgaaaaatggtcattctgaatcttcaggactaatacatctggaaggtgatactgtacaaattatttgcaacacaggatacagccttcaaaacaatgagaaaaacatttcgtgtgtagaacggggctggtccactcctcccatatgcagcttcactaaaggagaatgtcatgttccaattttagaagccaatgtagatgctcagccaaaaaaagaaagctacaaagttggagacgtgttgaaattctcctgcagaaaaaatcttataagagttggatcagactcagttcaatgttaccaatttgggtggtcacctaactttccaacatgcaaaggacaagtacgatcatgtggtccacctcctcaactctccaatggtgaagttaaggagataagaaaagaggaatatggacacaatgaagtagtggaatatgattgcaatcctaattttataataaacgggcctaagaaaatacaatgtgtggatggagaatggacaactttacccacttgtgttgaacaagtgaaaacatgtggatacatacctgaactcgagtacggttatgttcagccgtctgtccctccctatcaacatggagtttcagtcgaggtgaattgcagaaatgaatatgcaatgattggaaataacatgattacctgtattaatggaatatggacagagcttcctatgtgtgttgcaacacaccaacttaagaggtgcaaaatagcaggagttaatataaaaacattactcaagctatctgggaaagaatttaatcataattctagaatacgttacagatgttcagacatcttcagatacaggcactcagtctgtataaacgggaaatggaatcctgaagtagactgcacagaaaaaagggaacaattctgcccaccgccacctcagatacctaatgctcagaatatgacaaccacagtgaattatcaggatggagaaaaagtagctgttctctgtaaagaaaactatctacttccagaagcaaaagaaattgtatgtaaagatggacgatggcaatcattaccacgctgtgttgagtctactgcatattgtgggccccctccatctattaacaatggagataccacctcattcccattatcagtatatcctccagggtcaacagtgacgtaccgttgccagtccttctataaactccagggctctgtaactgtaacatgcagaaataaacagtggtcagaaccaccaagatgcctagatccatgtgtggtatctgaagaaaacatgaacaaaaataacatacagttaaaatggagaaacgatggaaaactctatgcaaaaacaggggatgctgttgaattccagtgtaaattcccacataaagcgatgatatcatcaccaccatttcgagcaatctgtcaggaagggaaatttgaatatcctatatgtgaatgaagcaagcataattttcctgaatatattcttcaaacatccatctatgctaaaagtagccattatgtagccaattctgtagttacttcttttattctttcaggtgttgtttaactcagttttatttagaactctggatttttagagctttagaaatttgtaagctgagagaacaatgtttcacttaataggagggtgtcttagtccatattacattgttataacagagtatcacagactggataacttctaaccaatagtttatttgtttcataaatctaaaagctgagaagtccaagatggtggggctgcctctggtgagggtcttctcgaagcatcataatatgctggaaggcatcacaacatggtggaagggatcacgtggcaaaagagcatgtacatgggagtgagagaaaaagagagagagagacagagtggcgggggcggggaggagcgcaaactcatcctttataaagacaccactcctgagataacaatccaatcccatgataatgacattaatccattcaagaagatagagctctcgtgacttaatcaccttctaaagatctcacctgacaacactgttgcattggcagttaagtttccacgtaaactttcggggacacattcaaaccacaggagaaactcaaattgttcctgggcaaatcacaacatggg